(19) United States
(12) Patent Application Publication
(10) Pub. No.: US 2011/0160239 A1
(43) Pub. Date: Jun. 30, 2011

(54) ORAL ADMINISTRATION OF PERIPHERALLY-ACTING OPIOID ANTAGONISTS

(76) Inventors: Kevin J. Brodbeck, Palo Alto, CA (US); Alan R. Kugler, Montara, CA (US)

(21) Appl. No.: 12/991,229

(22) PCT Filed: May 7, 2009
(86) PCT No.: PCT/US09/02856

§ 371 Date: Jan. 14, 2011

Related U.S. Application Data
(60) Provisional application No. 61/126,868, filed on May 7, 2008.

(51) Int. Cl. A61K 31/485; A61P 1/10; A61P 25/04

(52) U.S. Cl. 514/282

(57) ABSTRACT

Peripherally-acting opioid antagonists can be orally administered to treat the side effects of opioid administration in convenient dosing schedules.

### CROSS REFERENCE TO RELATED APPLICATION

This application claims the benefit of priority under 35 U.S.C. §119(e) to U.S. Provisional Patent Application Ser. No. 61/126,868, filed 7 May 2008, the disclosure of which is incorporated herein by reference in its entirety.

### FIELD OF THE INVENTION

The present invention relates to methods for inhibiting the peripheral action of opioids by administering therapeutically effective doses of peripherally acting opioid antagonists. The invention relates to the fields of pharmacology and medicine.

### BACKGROUND OF THE INVENTION

Through their actions on receptors in the central nervous system, exogenous opioids effectively relieve pain; however, opioids also act on receptors in the enteric nervous system, thereby disrupting normal gastrointestinal function. See Panchal et al. (2007) Int J Clin Pract. 61(7):1181-1187 and Thomas (2008) J Pain Symptom Manage 35(1):103-113. Constipation is a common and potentially debilitating adverse effect associated with opioid use. Depending on the population studied and the definitions used, constipation occurs in 15% to 90% of patients taking opioids. See Panchal et al. (2007). Opioid-induced constipation (OIC) significantly impacts a patient's quality of life and increases healthcare utilization; patients with OIC visit a physician significantly more often than opioid-treated patients without OIC. See Bell et al. (2007) J Pain. 8(4):S75, Abstract 897 and Eldon et al. (2007) Poster presented at the Annual Meeting of the American Academy of Pain Management; Las Vegas, Nev., September 27-30, Poster 28. While constipation is generally the predominant component of opioid-induced bowel dysfunction (OBD), patients taking opioids may experience a spectrum of other troublesome gastrointestinal effects, including gastroesophageal reflux, abdominal cramping, and bloating. See Panchal et al. (2007).

Naloxone is a drug used to counter the effects of opioid overdose, such as heroin or morphine overdose, specifically to counteract life-threatening depression of the central nervous system and respiratory system. Naloxone is marketed under various trademarks including Narcan, Nalone, and Narcanti. Naloxone cannot be used to treat the side effects of opioid administration without, however, counteracting the analgesic effect of the opiod as well.

Methylnatrexone (RELISTOR®, Wyeth Pharmaceuticals Inc., Philadelphia Pa.) and alvimopan are opioid antagonists with activity restricted to peripheral gut receptors. Both drugs have the ability to reverse opioid-induced ileus without reversing analgesia. Alvimopan can be administered orally, and it is not absorbed through the gastric mucosa. Methylnaltrexone, a quaternary derivate of naltrexone, does not cross the blood-brain barrier and acts as a selective peripheral opioid receptor antagonist.

Polyethylene glycol-conjugated naloxol (PEG-naloxol) compounds are chemical derivatives of the opioid antagonist naloxone that also act as peripheral opioid antagonists of opioid receptors within the enteric nervous system (see U.S. Patent Application Publication Nos. 2005/0136031 and 2006/0105046 and PCT Patent Application Publication Nos. WO 2007/124114 and WO 2008/057579, each of which is incorporated herein by reference). PEGylation (which has been described as the chemical derivatization of a compound by conjugation of one or more PEG moieties) impedes penetration of the derivatized compound, relative to the underivatized compound, across the blood brain barrier, as has been demonstrated in an animal models. See Eldon et al. (2007) supra. In preclinical studies, PEG-naloxol improved gastrointestinal transit time while maintaining central analgesia in a rodent model of morphine-induced constipation. Id. In a proof-of-principle phase 1 trial, single oral doses of a peripherially acting opioid antagonist antagonized morphine-induced delay in gastrointestinal transit time but preserved central opioid effects, as measured by pupillometry. See Neumann et al. (2007) Poster presented at the Annual Meeting of the American Academy of Pain Management; Las Vegas, Nev., September 27-30, Poster 27.

While the advent of peripherally acting opioid antagonists offers great promise for the treatment of the side effects associated with opioid use, there remains a need for new dosage forms and methods of administration of these promising agents that can enable them to be used to the greatest therapeutic effect. The present invention meets these and other needs.

### SUMMARY OF THE INVENTION

In one or more embodiments of the invention, a method is provided, the method comprising orally administering to an individual a therapeutically effective dose of a peripherally acting opioid antagonist no more than twice daily.

In one or more embodiments of the invention, a method for treating or preventing one or more opioid-induced side effects (e.g., opioid-induced bowel dysfunction) in a patient treated with an opioid without significant inhibition of the central analgesic effect of said opioid, said method comprising orally administering a therapeutically effective dose of a peripherally acting opioid antagonist no more than twice daily, preferably wherein said dose provides a therapeutic benefit (e.g., treatment or prevention of opioid-induced bowel dysfunction) for at least ten hours.

In one or more embodiments of the invention, a method is provided, the method comprising orally administering to an individual a therapeutically effective dose of a peripherally acting opioid antagonist, wherein the peripherally acting opioid antagonist is administered only once per day.

In one or more embodiments of the invention, a method is provided, the method comprising orally administering to an individual a therapeutically effective dose of a peripherally acting opioid antagonist, wherein the peripherally acting opioid antagonist is selected from the group consisting of methylnatrexone, alvimopan, and a compound encompassed by Formula I described herein.

In one or more embodiments of the invention, a method is provided, the method comprising orally administering to an individual a therapeutically effective dose of a peripherally acting opioid antagonist, wherein the therapeutically effective dose is a dose within one or more of the following ranges: 5 mg to 100 mg per day; 10 mg to 100 mg per day; 25 mg to 100 mg per day; and 5 mg to 50 mg per day.

In one or more embodiments of the invention, a unit dose form of a pharmaceutical formulation of an orally administrable opioid antagonist that provides a therapeutic benefit for at least 10 hours to a patient taking an opioid, wherein the unit dose form is administered for the treatment or prevention of opioid-induced bowel dysfunction without significant inhibition of the central analgesic effect of said opioid.

In one or more embodiments of the invention, a unit dose form is provided, the unit dose form comprising a therapeutically effective dose of an opioid and a therapeutically effective dose of a peripherally acting opioid antagonist. In one or more embodiments of the invention, the unit dose form comprises said peripherally acting opioid antagonist in an amount such that, upon administration of the unit dose form to an individual, significant inhibition of the central analgesic effect of said opioid occurs in an individual receiving an overdose of said unit dose form. In one or more embodiments of the invention, the unit dose form comprises said peripherally acting opioid antagonist in an amount such that, upon administration of the unit dose form, significant inhibition of the central analgesic effect of said opioid occurs in an individual injecting a liquefied form (such as a suspension or a solution) of said unit dose form.

In one or more embodiments of the invention, a method for inducing a bowel movement in an opioid-treated individual suffering from opioid-induced constipation without significant inhibition of the central analgesic effect of the opioid in said individual, said method comprising orally administering a therapeutically effective dose of a peripherally acting opioid antagonist, wherein said opioid antagonist preferably reaches its Cmax in said individual within 3 hours of said administering step.

In one or more embodiments of the invention, a method for treating or preventing opioid-induced bowel dysfunction in an individual treated with an opioid without significant inhibition of the central analgesic effect of said opioid in said individual, said method comprising orally administering a therapeutically effective dose of a peripherally acting opioid antagonist, preferably sufficient to provide area under the curve from 0 to 12 hours in the range of 140 hours×ng/mL to 1300 hours×ng/mL.

In one or more embodiments of the invention, the invention provides an orally administrable, peripherially acting opioid antagonist having a half-life in humans of greater than 10 hours.

### BRIEF DESCRIPTION OF THE DRAWING

FIG. 1. is a graph showing the mean (±SEM) plasma COMPOUND 1 concentration-time profiles for Day 8, all treatments, log-linear scale (n=6)

### DETAILED DESCRIPTION OF THE INVENTION

Before describing the present invention in detail, it is to be understood that this invention is not limited to the active agents specifically set forth herein, as such active agents are examples of active agents that are encompassed by the invention. For example, other active agents not currently known but possess the same features recited in the claims herein are also encompassed by the invention.

It must be noted that, as used in this specification and the claims, the singular forms “a,” “an,” and “the” include plural referents unless the context clearly dictates otherwise.

In describing and claiming the present invention, the following terminology will be used in accordance with the definitions described below.

“PEG,” “polyethylene glycol” and “poly(ethylene glycol)” as used herein, are meant to encompass any water-soluble poly(ethylene oxide). Typically, PEGs for use in the pharmaceutical context comprise the following structure “—O(CH₂CH₂O)ₘ—” where (m) is 2 to 4000. As used herein, PEG also includes “—CH₂CH₂—O(CH₂CH₂O)ₘ—CH₂CH₂—” and “—(CH₂CH₂O)ₘ—,” depending upon whether or not the terminal oxygens have been displaced. When the PEG further comprises a spacer moiety (to be described in greater detail below), the atoms comprising the spacer moiety, when covalently attached to a water-soluble polymer segment, do not result in the formation of an oxygen-oxygen bond (i.e., an “—O—O—” or peroxide linkage). Throughout the specification and claims, it should be remembered that the term “PEG” includes structures having various terminal or “end capping” groups and so forth. The term “PEG” also means a polymer that contains a majority, that is to say, greater than 50%, of —CH₂CH₂O— monomeric subunits. With respect to specific forms, the PEG can take any number of a variety of molecular weights, as well as structures or geometries such as “branched,” “linear,” “forked,” “multifunctional,” and the like, to be described in greater detail below.

An “organic radical” as used herein includes, for example, alkyl, substituted alkyl, alkenyl, substituted alkenyl, alkynyl, substituted alkynyl, aryl and substituted aryl.

“Alkyl” refers to a hydrocarbon chain, typically ranging from about 1 to 20 atoms in length. Such hydrocarbon chains are preferably but not necessarily saturated and may be branched or straight chain, although typically straight chain is preferred. Exemplary alkyl groups include ethyl, propyl, butyl, pentyl, 1-methylbutyl, 1-ethylpropyl, 3-methylpentyl, and the like. As used herein, “alkyl” includes cycloalkyl when three or more carbon atoms are referenced and lower alkyl.

“Lower alkyl” refers to an alkyl group containing from 1 to 6 carbon atoms, and may be straight chain or branched, as exemplified by methyl, ethyl, n-butyl, iso-butyl, and tert-butyl.

“Cycloalkyl” refers to a saturated or unsaturated cyclic hydrocarbon chain, including bridged, fused, or Spiro cyclic compounds, preferably made up of 3 to about 12 carbon atoms, more preferably 3 to about 8.

The term “substituted” as in, for example, “substituted alkyl,” refers to a moiety (e.g., an alkyl group) substituted with one or more non-interfering substituents, such as, but not limited to: C₃-C₈ cycloalkyl, e.g., cyclopropyl, cyclobutyl, and the like; halo, e.g., fluoro, chloro, bromo, and iodo; cyano; alkoxy, lower phenyl (e.g., 0-2 substituted phenyl); substituted phenyl; and the like, for one or more hydrogen atoms.

As used herein, “alkenyl” refers to a branched or unbranched hydrocarbon group of 1 to 15 atoms in length, containing at least one double bond, such as ethenyl, n-propenyl, isopropenyl, n-butenyl, isobutenyl, octenyl, decenyl, tetradecenyl, and the like.

The term “alkynyl” as used herein refers to a branched or unbranched hydrocarbon group of 2 to 15 atoms in length, containing at least one triple bond, ethynyl, n-butynyl, isopentynyl, octynyl, decynyl, and so forth.

“Pharmaceutically acceptable excipient” or “pharmaceutically acceptable carrier” refers to an excipient that can be included in the compositions of the invention and that causes no significant adverse toxicological effects to the individual (i.e., patient).

“Therapeutically effective amount” refers to the amount of an active agent (e.g., a peripherally acting opioid antagonist and a opioid agonist) that is needed to provide a desired level of active agent in the bloodstream or in a target tissue. The exact amount will depend upon numerous factors, e.g., the particular active agent, the components and physical characteristics of the pharmaceutical preparation, intended patient population, patient considerations, and the like, and can readily be determined by one of ordinary skill in the art, based upon the information provided herein.

The terms “patient” and “individual” are interchangeable and refer to a living organism suffering from or prone to a condition that can be prevented or treated by administration of a peripherally acting opioid antagonist, and includes both humans and animals. As used herein, it will be understood that reference to a central analgesic effect means the central analgesic effect associated within an opioid-treated individual (i.e., an individual receiving opioid-based analgesia via the administration of one or more opioid analgesics).

“Optional” and “optionally” mean that the subsequently described circumstance may or may not occur, so that the description includes instances where the circumstance occurs and instances where it does not.

As previously indicated, the present invention provides (among other things) various methods that comprise orally administering a peripherally acting opioid antagonist to a patient. Typically, the patient has already received opioid-based therapies via the administration of one or more opioid analgesic to provide the patient with a central analgesic effect, although instances wherein the opioid-based therapy is initiated concomitantly or subsequently to oral administration of the peripherally acting opioid antagonist are also contemplated.

Exemplary peripherally acting opioid antagonists include compounds encompassed wherein a water-soluble oligomer is covalently attached to a moiety having antagonism at opioid receptors. See, for example, the compounds disclosed in U.S. Patent Application Publication No. 2003/0124086.

Still further compounds Such compounds include, by way of example only, those encompassed by Formula I, below.

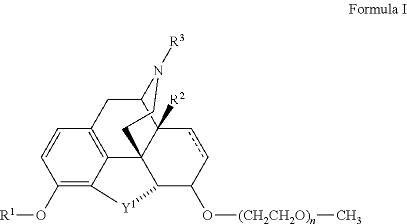

wherein:

R¹ is H or an organic radical (preferably H);

R² is H or OH (preferably OH);

R³ is H or an organic radical (preferably R³ is H or an organic radical such as C₁₋₆ alkyl, substituted C₁₋₆ alkyl, C₃₋₆ cycloalkyl, substituted C₃₋₆ cycloalkyl, C₂₋₆ alkenyl, substituted C₂₋₆ alkenyl, C₂₋₆ alkynyl, substituted C₂₋₆ alkynyl, and more preferably CH₂—CH═CH₂);

the dotted line (“- - -”) represents an optional double bond;

Y¹ is O or S (preferably O); and

(n) is an integer from 3 to 20 (preferably from 3 to 10),

and all stereoisomers thereof as well as pharmaceutically acceptable salts of all of the foregoing.

A preferred peripherally acting opioid antagonist is COMPOUND I, which is a compound having the following formula:

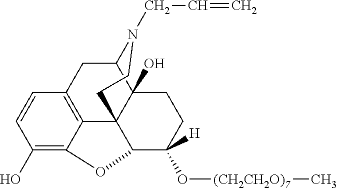

Exemplary ranges of half-lives of the peripherally acting opioid antagonist include: greater than 8 hours; greater than 9 hours; greater than 10 hours; greater than 11 hours; greater than 8 hours and less than 24 hours; greater than 10 hours and less than 24 hours; greater than 11 hours and less then 24 hours.

To achieve a central analgesic effect, the patient will typically be administered an opioid agonist. The opioid agonist can be administered to the patient by any suitable means, including, for example, by injection (including without limitation intravenously, intraarterially, subdermally, intraperitoneally, intramuscularly, and subcutaneously), orally, buccally, nasally, transmucosally, topically, via an ophthalmic preparation, and by inhalation. Administration of the opioid agonist can be achieved via self administration by the individual as well as by another. The therapeutically effective dose (including its frequency of dosing) of the opioid agonist will typically be in accordance with conventional administration schemes associated with the specific opioid and available, for example, in Drug Facts and Comparisons (2003) 57^{th} Edition, Kenneth Killion, Ed., Facts and Comparison, St. Louis, Mo.

The “opioid agonist” is any natural or synthetic alkaloid or structural derivative of opium that activates one or more opioid receptor types, including partial agonists (i.e., compounds exhibiting activity against less than all opioid receptor types) and agonist-antagonists (i.e., compounds exhibiting agonist activity at one receptor type and antagonist activity at another receptor type). The opioid agonist can be a natural alkaloid such as a penanthrene (e.g., morphine) or benzylisoquinoline (e.g., papaverine), a semi-synthetic derivative (e.g., hydromorphone), or any of various classes of synthetic derivatives (e.g., phenylpiperidines, benzmorphans, priopionanilides, and morphinans). Exemplary opioid agonists include 1-α-acetylmethadol, alfentanil, alphaprodine, anileridine, bremazocine, buprenorphine, butorphanol, codeine, cyclazocine, dezocine, diacetylmorphine (i.e., heroin), dihydrocodeine, ethylmorphine, fentanyl, hydrocodone, hydromorphone, levorphanol, meperidine (i.e., pethidine), methadone, methotrimeprazine, morphine, nalbuphine, nefopam, normophine, noscapine, oxycodone, oxymorphone, papaverine, pentazocine, pethidine, phenazocine, propiram, propoxyphene, sufentanil, thebaine and tramadol, and pharmaceutically acceptable salts of each of the foregoing. Structures of preferred opioid agonists are provided below:

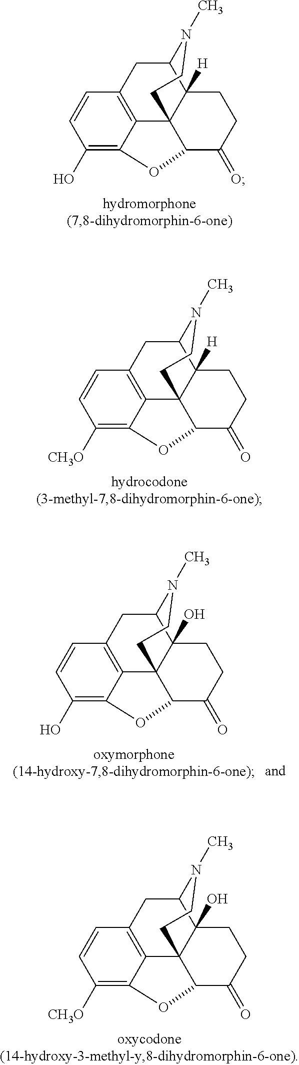

As previously stated, while exogenous opioids provide a patient the benefit of analgesia, they very often simultaneously result in peripheral side effects. Through orally administering a peripherally acting opioid antagonist, the benefits of both convenience (e.g., not having to administer an injection) as well as reversal of one or more opioid-induced side effects may be achieved. For example, in one embodiment, a method of the invention can be used in patients suffering from opioid-induced bowel dysfunction. In another exemplary embodiment, a method of the invention can be used in a patient undergoing opioid therapy in which inducement of a bowel movement is indicated. In all instances, preferred patients are human patients.

For oral delivery of a peripherally acting opioid antagonist, it is preferred that the dosage form is in the form of a unit dose form. In some embodiments of the present invention, the unit dose form comprises both the peripherally acting opioid antagonist and the opioid agonist.

In still other embodiments, the unit dose form will comprising both the peripherally acting opioid antagonist and the opioid agonist, wherein the opioid antagonist is present in an amount such that significant inhibition of the central analgesic effect of said opioid occurs in an individual injecting a liquefied form of said unit dose form. In this way, the abuse potential of the unit dose form may be minimized. While not wishing to be bound by theory, peripherally acting opioid antagonists—when present in sufficient and relatively high amounts—may overwhelm the blood-brain barrier filtering mechanism and subsequently penetrate into the central nervous system. Upon entering the central nervous system, the opioid antagonist can counteract the effects of the opioid agonist and thereby frustrate the addict's attempt to abuse the opioid agonist.

For orally administered drugs, including the peripherally acting opioid antagonist (as well as the opioid agonist if the oral route is used) suitable oral unit dose forms can be in the form of a liquid, semi-solid or solid. Exemplary liquids include a suspension, a solution, an emulsion, and a syrup. Exemplary semi-solids include gels which can be administered “as is” or formulated (e.g., into a gel-cap) for administration to a patient. Exemplary solids include granules, pellets, beads, powders, which can be administered “as is” or formulated into one or more of the following for administration to a patient: a tablet; a capsule; a caplet; gel cap and troche. Suitable pharmaceutical compositions and unit dose forms may be prepared using conventional methods known to those in the field of pharmaceutical formulation and described in the pertinent texts and literature, e.g., in Remington's Pharmaceutical Sciences: 18^{th} Edition, Gennaro, A. R., Ed. (Mack Publishing Company; Easton, Pa.; 1990).

Tablets and capsules represent the most convenient oral dosage forms. Tablets can be manufactured using standard tablet processing procedures and equipment. Preferred techniques for forming tablets include direct compression and granulation. In addition to the active agents, tablets will generally contain inactive, pharmaceutically acceptable carrier materials such as binders, lubricants, disintegrants, fillers, stabilizers, surfactants, coloring agents, and the like. Binders are used to impart cohesive qualities to a tablet, and thus ensure that the tablet remains intact. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate, calcium stearate, and stearic acid. Disintegrants are used to facilitate disintegration of the tablet, and are generally starches, clays, celluloses, aligns, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers, as well known in the art, are used to inhibit or retard drug decomposition reactions that include, by way of example, oxidative reactions.

In some instances, the tablet can be in the form of a uniform tablet. In uniform tablets, the formulation used in preparing the tablet is a substantially homogenous mixture of active agents and one or more pharmaceutical excipient (e.g., diluent). The formulation is then used to make tablets using a suitable tableting process to thereby result in a tablet that is substantially homogenous throughout the tablet.

In still other instances, the tablet can also take the form of a layered tablet (of one, two, three or more layers). The method for manufacturing the layered tablet can include combining two different formulations (e.g., one formulation containing the opioid agonist and another containing the polymer-opioid conjugate) and compressing the two together to form the tablet. Multiple layered tablets of three or more layers are also possible and can be formed, for example, in a similar manner by combining three or more distinct formulations and followed by compression.

Optionally, a barrier layer can be included in the layered tablet. One approach for incorporating a barrier layers involves forming a compressed first layer of a first formulation (e.g., a formulation containing a first active agent) wherein the compress layers has one exposed surface, coating the exposed surface with a material (e.g., a material that is substantially impermeable to thereby prevent physical interaction between adjacent layers) to form a coated surface, and contacting the coated surface with a second formulation (e.g., a second formulation containing a second active agent), and compressing the second formulation and coated surface to form a layered tablet having a barrier layer included therein.

Capsules are also preferred oral dosage forms, in which case the composition may be encapsulated in the form of a liquid, semi-solid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington's Pharmaceutical Sciences, supra, which describes materials and methods for preparing encapsulated pharmaceuticals.

Exemplary excipients include, without limitation, those selected from the group consisting of carbohydrates, inorganic salts, antimicrobial agents, antioxidants, surfactants, buffers, acids, bases, and combinations thereof.

A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, sorbitol (glucitol), pyranosyl sorbitol, myoinositol, and the like.

The excipient can also include an inorganic salt or buffer such as citric acid, sodium chloride, potassium chloride, sodium sulfate, potassium nitrate, sodium phosphate monobasic, sodium phosphate dibasic, and combinations thereof.

The preparation may also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the preparation as well. Antioxidants are used to prevent oxidation, thereby preventing the deterioration of the conjugate or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant may be present as an excipient. Exemplary surfactants include: polysorbates, such as “Tween 20” and “Tween 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and chelating agents, such as EDTA, zinc and other such suitable cations.

Acids or bases may be present as an excipient in the preparation. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The pharmaceutical preparations encompass all types of formulations. The amount of the active agents (i.e., opioid agonist and the polymer-opioid antagonist conjugate) in the composition will vary depending on a number of factors, but will optimally be a therapeutically effective dose of each active agent when the composition is stored in a unit dose form. A therapeutically effective dose for each active agent can be determined experimentally by repeated administration of increasing amounts of the active agent in order to determine which amount produces a clinically desired endpoint as determined by a clinician.

The amount of any individual excipient in the composition will vary depending on the activity of the excipient and particular needs of the composition. Typically, the optimal amount of any individual excipient is determined through routine experimentation, i.e., by preparing compositions containing varying amounts of the excipient (ranging from low to high), examining the stability and other parameters of the composition, and then determining the range at which optimal performance is attained with no significant adverse effects.

Generally, however, the excipient will be present in the composition in an amount of about 1% to about 99% by weight, preferably from about 2%-98% by weight, more preferably from about 5-95% by weight of the excipient, with concentrations less than 30% by weight most preferred.

These foregoing pharmaceutical excipients along with other excipients are described in “Remington: The Science & Practice of Pharmacy”, 19^{th} ed., Williams & Williams, (1995), the “Physician's Desk Reference”, 52′^{d} ed., Medical Economics, Montvale, N.J. (1998), and Kibbe, A. H., Handbook of Pharmaceutical Excipients, 3^{rd} Edition, American Pharmaceutical Association, Washington, D.C., 2000.

It is to be understood that while the invention has been described in conjunction with the preferred specific embodiments thereof, that the foregoing description as well as the experimental that follow are intended to illustrate and not limit the scope of the invention.

Other aspects, advantages and modifications within the scope of the invention will be apparent to those skilled in the art to which the invention pertains.

All articles, books, patents, patent publications and other publications referenced herein are hereby incorporated by reference in their entireties.

### EXPERIMENTAL

As used in the Example 1, COMPOUND 1 refers to a compound having the structure provided below.

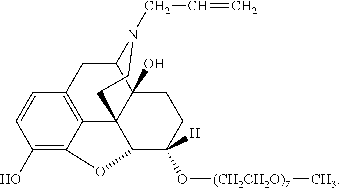

COMPOUND I can be prepared as described in U.S. Patent Application Publication Nos. 2005/0136031, 2006/0105046 and PCT Patent Application No. WO 2007/124114.

### Example 1

A double-blind, randomized, placebo-controlled, multiple-dose study was conducted to evaluate the safety, tolerability, and pharmacokinetics of oral doses of COMPOUND I.

Thirty-two healthy male and female volunteers were enrolled in this randomized, double-blind, placebo-controlled, multiple-dose, dose-escalation study. The main inclusion criteria were: (i) aged≧18 and ≦65 years; (ii) body mass index (BMI)≧18 and ≦30 kg/m²; (iii) nonsmokers without a history of drug or alcohol abuse; (iv) normal bowel movement frequency during the past month; and (v) female subjects had to be postmenopausal or surgically sterilized. There were 16 male and 16 female subjects who participated in the study. Subjects ranged in age from 25 to 65 years. BMI (weight in kilograms divided by height in meters squared) ranged from 19 to 29.

Subjects were randomized 3:1 to COMPOUND I oral solution or placebo oral solution twice daily (every 12 hours) for 7 days (with a single dose on the eighth day). Subjects were assigned to one of four cohorts: 25 mg, 60 mg, 125 mg, or 250 mg twice daily. Each cohort consisted of eight subjects; six were treated with active drug and two received placebo. Each cohort included four male and four female subjects. Subjects did not receive opioid therapy during the study. Safety was assessed by monitoring adverse events, vital signs, electrocardiogram recordings, and clinical laboratory parameters, including hematology, serum biochemistry, and urinalysis.

Blood samples were collected for measurement of plasma COMPOUND I and COMPOUND I-glucuronide concentrations via a validated LC-MS/MS method. Individual and mean plasma COMPOUND I and COMPOUND I-glucuronide concentrations as a function of sampling time were plotted on linear and log-linear scales. Individual pharmacokinetic parameters were derived by noncompartmental analysis and summarized by treatment. Attainment of steady-state, dose-proportionality, and gender comparisons were evaluated graphically.

There were no deaths, serious adverse events, or premature study discontinuations. In general, adverse event rates were similar in the placebo and treatment groups; six of eight subjects (75%) in the placebo group and 18 of 24 (75%) in the treatment groups experienced at least one adverse event. Tables 1 and 2 summarize the treatment-emergent adverse events observed in the study.

A drug-related adverse event was defined as an adverse event that was considered “possibly related” or “definitely related” to study drug in the opinion of the investigator; there were no drug-related adverse events that were deemed as definitely related to study drug. The majority of the drug-related adverse events were of mild intensity; of 69 drug-related adverse events, 62 (90%) were rated as mild and 7 (10%) were rated as moderate. Adverse events did not appear to be dose related, with the possible exception of dizziness. No subject in the 25- or 60-mg dose groups experienced dizziness. Two of six subjects in the 125-mg group and three of six subjects in the 250-mg group experienced dizziness. However, two of eight subjects in the placebo group also experienced dizziness. No clinically significant drug-related laboratory toxicities or electrocardiographic changes were observed.

TABLE 1
Summary of Treatment-Emergent Adverse Events

| | Subjects | Subjects | Total number |
|---|---|---|---|
| | with adverse | with adverse | of adverse |
| Dose group | events, n | events, % | events |
| Placebo (n = 8) | 6 | 75 | 29 |
| 25 mg Q12H (n = 6) | 4 | 66.7 | 7 |
| 60 mg Q12H (n = 6) | 5 | 83.3 | 13 |
| 125 mg Q12H (n = 6) | 4 | 66.7 | 14 |
| 250 mg Q12H (n = 6) | 5 | 83.3 | 23 |

Q12H, every 12 hours.

TABLE 2
Treatment-Emergent Adverse Events Occurring in More Than 1 Subject*

| | Placebo | | 25 mg Q12H | | 60 mg Q12H | | 125 mg Q12H | | 250 mg Q12H | |
|---|---|---|---|---|---|---|---|---|---|---|
| | (N = 8) | | (N = 6) | | (N = 6) | | (N = 6) | | (N = 6) | |
| Adverse Event | Events | N | Events | n | Events | n | Events | N | Events | n |
| Abdominal pain | 1 | 1 | — | — | 2 | 2 | — | — | 1 | 1 |
| Constipation | — | — | — | — | — | — | 1 | 1 | 1 | 1 |
| Diarrhea | 1 | 1 | — | — | — | — | 1 | 1 | — | — |
| Discolored feces | 2 | 2 | 1 | 1 | 1 | 1 | — | — | — | — |
| Flatulence | 1 | 1 | — | — | 5 | 5 | 2 | 2 | — | — |
| Nausea | 2 | 2 | — | — | 1 | 1 | 1 | 1 | 2 | 2 |
| Catheter site | — | — | — | — | — | — | — | — | 2 | 2 |
| pain | | | | | | | | | | |
| Catheter site- | 1 | 1 | — | — | — | — | 1 | 1 | — | — |
| related reaction | | | | | | | | | | |
| Back pain | 1 | 1 | — | — | — | — | 1 | 1 | 1 | 1 |
| Myalgia | — | — | 1 | 1 | — | — | 2 | 1 | 2 | 2 |
| Dizziness | 4 | 2 | — | — | — | — | 2 | 2 | 5 | 3 |
| Headache | 7 | 2 | 1 | 1 | 2 | 2 | 2 | 2 | 1 | 1 |

*Events, number of events reported; n, number of subjects reporting event.
Q12H, every 12 hours.

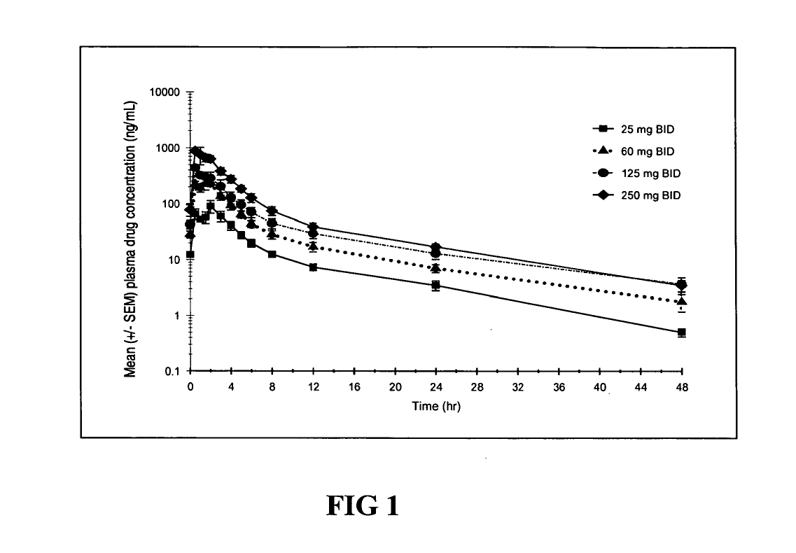

COMPOUND I was rapidly absorbed, as evidenced by a steep increase of plasma COMPOUND I concentration at all dose levels. Secondary COMPOUND I concentration-time profile peaks or shoulders following the initial peak were frequently observed, especially at lower doses. Maximum COMPOUND I plasma concentration (Cₘₐₓ) and area under the plasma COMPOUND I concentration-time curve (AUC) values were linear (dose-proportional) on Day 1 and Day 8 of dosing (Tables 3 and 4). Multi-phasic kinetics were evident from the plasma COMPOUND I concentration-time profiles on Day 8 (FIG. 1).

TABLE 3
Primary Plasma COMPOUND I Pharmacokinetic
Parameters, Day 1

| | Mean (SD) | | |
|---|---|---|---|
| | Cₘₐₓ | tₘₐₓ | AUC₀₋₁₂ |
| Dose group | (ng/mL) | (h) | (h × ng/mL) |
| 25 mg Q12H (n = 6) | 76.9 (37.59) | 1.58 (0.97) | 248.0 (78.32) |
| 60 mg Q12H (n = 6) | 242.7 (112.4) | 0.75 (0.61) | 531.8 (239.8) |
| 125 mg Q12H (n = 6) | 324.8 (84.73) | 0.83 (0.61) | 996.0 (292.5) |
| 250 mg Q12H (n = 6) | 990.7 (492.8) | 0.50 (0.00) | 1974 (700.9) |

AUC₀₋₁₂, area under plasma COMPOUND I concentration-time curve from 0 to 12 hours; Cₘₐₓ, maximum COMPOUND I plasma concentration; Q12H, every 12 hours; SD, standard deviation; Tₘₐₓ, time to maximum plasma COMPOUND I concentration.

TABLE 4
Primary Plasma COMPOUND I Pharmacokinetic Parameters, Day 8

| | Mean (SD) | | | |
|---|---|---|---|---|
| | Cₘₐₓ | tₘₐₓ | AUC₀₋₁₂ | t_{1/2Z} |
| Dose group | (ng/mL) | (h) | (h × ng/mL) | (h) |
| 25 mg Q12H (n = 6) | 96.87 (55.38) | 1.92 (0.80) | 363.9 (151.0) | 9.389 (2.044) |
| 60 mg Q12H (n = 6) | 288.2 (102.9) | 1.42 (0.74) | 961.1 (323.2) | 10.96 (4.176) |
| 125 mg Q12H (n = 6) | 489.7 (112.8) | 0.75 (0.61) | 1457 (588.6) | 11.67 (3.111) |
| 250 mg Q12H (n = 6) | 1054 (364.1) | 0.69 (0.30) | 2985 (1057) | 10.52 (2.497) |

AUC₀₋₁₂, area under plasma COMPOUND I concentration-time curve from 0 to 12 hours; Cₘₐₓ, maximum COMPOUND I plasma concentration; Q12H, every 12 hours; SD, standard deviation; Tₘₐₓ, time to maximum plasma COMPOUND I concentration; T_{1/2Z}, terminal plasma COMPOUND I half-life.

The observed terminal COMPOUND I half-life was approximately 11 hours, independent of dose. Steady-state was generally reached within a few doses. Plasma COMPOUND I—glucuronide concentrations were approximately 100-fold less than plasma COMPOUND I concentrations. Glucuronidation was not affected by dose level or duration of dosing.

These results demonstrate that oral COMPOUND I is safe and generally well tolerated at doses up to 250 mg twice daily, with no serious or severe adverse events, and no discontinuations for toxicity. COMPOUND I appeared rapidly in plasma after dose administration, demonstrating its bioavailability as an oral drug; pharmacokinetics were linear (dose-proportional), and the observed terminal plasma COMPOUND I half-life was approximately 11 hours, independent of dose.

The results also demonstrate that orally administered, peripherally-acting opioid antagonists can be administered in therapeutically effective doses for the treatment for OIC and other manifestations of OBD. Thus, the present invention provides a method for treating or preventing opioid-induced bowel dysfunction in a patient treated with an opioid without significant inhibition of the central analgesic effect of said opioid, said method comprising orally administering a therapeutically effective dose of a peripherally acting opioid antagonist no more than twice daily, wherein said dose provides therapeutic benefit for at least ten hours each day. As the results above demonstrate, COMPOUND I has a serum half-life of about 11 hours and can be administered safely at relatively high doses. Thus, in one embodiment of the invention, in which the antagonist is COMPOUND I or a similar PEG-opioid antagonist, the therapeutically effective dose is in a range of 25 mg to 250 mg per day (and even lower doses, e.g., 5 mg, 10 mg, 12.mg, 15 mg, and 20 mg per day, can also be effective), which may be administered once daily or divided into two or more doses administered throughout the day (such as, for example, on the same dosing schedule as the opioid being administered to the patient). In various embodiments, the daily dose is 5, 10, 12, 15, 20, 25, 50, and 100 mg per day. Dose amounts can be adjusted accordingly for PEG-opioid antagonist compounds that differ significantly from COMPOUND I in molecular weight/bioavailability/activity, etc.

The present invention also provides unit dose forms of a pharmaceutical formulation of an orally administrable opioid antagonist that provides at least 10 hours of therapeutic benefit to a patient taking an opioid, wherein said therapeutic benefit is the treatment or prevention of opioid-induced bowel dysfunction without significant inhibition of the central analgesic effect of said opioid. In one embodiment, the antagonist is selected from the group consisting of methylnatrexone, alvimopan, and PEG-opioid antagonist. In one embodiment, the antagonist is COMPOUND I or a similar PEG-opioid antagonist, and the therapeutically effective dose is in a range of 25 mg to 250 mg per day (and even lower doses, e.g., 5 mg, 10 mg, 12.mg, 15 mg, and 20 mg per day, can also be effective), which may be administered once daily or divided into two or more doses administered throughout the day (such as, for example, on the same dosing schedule as the opioid being administered to the patient). In various embodiments, the therapeutically effective dose is 5, 10, 12, 15, 20, 25, 50, and 100 mg per day. Dose amounts can be adjusted accordingly for PEG-opioid antagonist compounds that differ significantly from COMPOUND I in molecular weight/bioavailability/activity, etc.

In another embodiment of the invention, the unit dose form further comprises a therapeutically effective dose of an opioid, optionally wherein said opioid antagonist is present in an amount such that significant inhibition of the central analgesic effect of said opioid occurs in an individual receiving an overdose of said unit dose form. In one embodiment, the opioid antagonist is present in an amount such that significant inhibition of the central analgesic effect of said opioid occurs in an individual injecting a liquefied form of said unit dose form. The dizziness experienced by some patients at the high doses tested in the study described above may be in part due to some penetration of the blood brain barrier by PEG-opioid antagonist at high doses. Thus, when a patient attempts to abuse an opioid antagonist/opioid combination unit dose form of the invention (for example, by liquefication and injection), the high doses of the antagonist absorbed should result in blood brain barrier penetration and concomitant blocking of the analgesic effect of the opioid, frustrating the purpose of the abuser and also providing a safer dose form of the opioid.

The results above also show that the present invention provides a method for inducing a bowel movement in a patient suffering from opioid-induced constipation without significant inhibition of the central analgesic effect of the opioid in said patient, said method comprising orally administering a therapeutically effective dose of a peripherally acting opioid antagonist, wherein said opioid antagonist reaches its Cmax in said patient within 3 hours of said administering step. In one embodiment, the antagonist is administered no more than twice per day. In one embodiment, the antagonist is administered only once per day. In one embodiment, the antagonist is selected from the group consisting of methylnatrexone, alvimopan, and PEG-opioid antagonist. In one embodiment, the antagonist is COMPOUND I or a similar PEG-opioid antagonist, and the therapeutically effective dose is in a range of 25 mg to 250 mg per day (and even lower doses, e.g., 5 mg, 10 mg, 12.mg, 15 mg, and 20 mg per day, can also be effective), which may be administered once daily or divided into two or more doses administered throughout the day (such as, for example, on the same dosing schedule as the opioid being administered to the patient). In various embodiments, the therapeutically effective dose is 5, 10, 12, 15, 20, 25, 50, and 100 mg per day. Dose amounts can be adjusted accordingly for PEG-opioid antagonist compounds that differ significantly from COMPOUND I in molecular weight/bioavailability/activity, etc. In one embodiment, the patient taking the opioid antagonist of the invention has 7 or more bowel movements per week, but in the absence of such treatment, has only 3 or fewer movements per week.

The present invention also provides a method for treating or preventing opioid-induced bowel dysfunction in a patient treated with an opioid without significant inhibition of the central analgesic effect of said opioid in said patient, said method comprising orally administering a therapeutically effective dose of COMPOUND I or a compound encompassed by Formula I sufficient to provide area under the curve from 0 to 12 hours values in the ranges shown in Tables 3 and 4, above, for the 25, 60, 125, and 250 mg dose groups.

These and other aspects and embodiments of the invention will be apparent to one of skill in the art upon contemplation of this disclosure.

What is claimed is:

1. A method comprising orally administering to an individual a therapeutically effective dose of a peripherally acting opioid antagonist no more than twice daily.

2. The method of claim 1, used in the treatment of an individual suffering from one or more peripherially mediated opioid-induced side effects.

3. The method of claim 1, used in the treatment of an individual suffering from opioid-induced bowel dysfunction.

4. The method of claim 1, used in the treatment of an individual suffering from opioid-induced constipation.

5. The method of claim 1, used in the prevention of one or more peripherially mediated opioid-induced side effects.

6. The method of claim 5, wherein a therapeutically effective dose of an opioid is administered to the individual within 24 hours of administering to the individual a therapeutically effective dose of a peripherally acting opioid antagonist.

7. The method of claim 1, wherein the peripherally acting opioid antagonist is administered once a day.

8. The method of claim 1, wherein the peripherally acting opioid antagonist is administered twice a day.

9. The method of claim 1, wherein the total daily dose of the peripherally acting opioid antagonist is from 10 mg to 100 mg.

10. The method of claim 9, wherein the total daily dose of the peripherally acting opioid antagonist is from 25 mg to 100 mg.

11. The method of claim 1, wherein the therapeutically effective dose of the peripherally acting opioid antagonist is from 5 mg to 50 mg.

12. The method of claim 1, wherein the therapeutically effective dose provides a therapeutic benefit for at least ten hours each day.

13. The method of claim 1, wherein the therapeutic benefit is selected from the group consisting of the prevention of an opioid-induced side effect in an individual and the treatment of an individual suffering from an opioid-induced side effect.

14. The method of claim 1, wherein the peripherally acting opioid antagonist has the following formula: 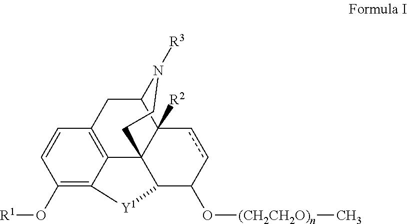
wherein:
R¹ is H or an organic radical;
R² is H or OH;
R³ is H or an organic radical;
the dotted line (“- - -”) represents an optional double bond;
Y¹ is O or S; and
(n) is an integer from 3 to 20,
and all stereoisomers thereof as well as pharmaceutically acceptable salts of all of the foregoing.

15. The method of claim 1, wherein the opioid is selected from the group consisting of 1-α-acetylmethadol, alfentanil, alphaprodine, anileridine, bremazocine, buprenorphine, butorphanol, codeine, cyclazocine, dezocine, diacetylmorphine, dihydrocodeine, ethylmorphine, fentanyl, hydrocodone, hydromorphone, levorphanol, meperidine, methadone, methotrimeprazine, morphine, nalbuphine, nefopam, normophine, noscapine, oxycodone, oxymorphone, papaverine, pentazocine, pethidine, phenazocine, propiram, propoxyphene, sufentanil, thebaine, tramadol and pharmaceutically acceptable salts of each of the foregoing.

16. A method comprising the steps of:
(i) administering to an individual a therapeutically effective amount of an opioid to provide a central analgesic effect;
(ii) orally administering, before, simultaneously with, or after step (i), to the individual a therapeutically effective amount of a peripherally acting opioid antagonist, wherein said dose of the peripherally acting opioid antagonist: (a) provides a therapeutic benefit for at least ten hours; and (b) does not cause significant inhibition of the central analgesic effect.

17. A unit dose form comprising a dose of an orally administrable opioid antagonist sufficient that, upon administration to an individual, provides a therapeutic benefit for at least 10 hours.

18. The unit dose form of claim 17, wherein the individual is experiencing a central analgesic effect.

19. The unit dose form of claim 18, wherein the unit dose form further comprises a therapeutically effective dose of an opioid.

20. The unit dose form of claim 17, wherein the individual is not experiencing a central analgesic effect.

21. The unit dose form of claim 17, wherein the therapeutic benefit is the treatment or prevention in an individual of opioid-induced side effect.

22. An orally administrable, peripherially acting opioid antagonist having a half-life in humans of greater than 10 hours.

23. A method comprising orally administering to an individual a therapeutically effective dose of a peripherally acting opioid antagonist, wherein said method induces a bowel movement in an individual receiving opioid therapy and suffering from opioid-induced constipation without significant inhibition of the central analgesic effect of the opioid in said individual.

24. A method comprising orally administering to an individual a therapeutically effective dose of a peripherally acting opioid antagonist, wherein said peripherally acting opioid antagonist reaches its Cmax in said individual within 3 hours of said administering.

25. A method comprising orally administering to an individual a therapeutically effective dose of a peripherally acting opioid antagonist, wherein said peripherally acting opioid antagonist provides an area under the curve from 0 to 12 hours following the administration in the range of 140 hours×ng/mL to 1300 hours×ng/mL.
